(19) United States
(12) Patent Application Publication
(10) Pub. No.: US 2003/0166063 A1
(43) Pub. Date: Sep. 4, 2003

(54) High level insect expression of rage proteins

(76) Inventors: Robert B. Harris, Midlothian, VA (US); Jane M. Shen, Winston-Salem, NC (US); Manouchehr Shahbaz, Escondido, CA (US)

Correspondence Address: Cynthia B. Rothschild, Esq., Kilpatrick Stockton LLP, 1001 West Fourth Street, Winston-Salem, NC, 27101, US

(21) Appl. No.: 10/091,019

(22) Filed: Mar. 5, 2002

Related U.S. Application Data
(60) Provisional application No. 60/273,418, filed on Mar. 5, 2001.

(51) Int. Cl. C12P 21/02; C12N 5/06; C12N 15/86

(52) U.S. Cl. 435/69.1; 435/456; 435/348; 435/320.1

(57) ABSTRACT

The present invention provides a method for high level expression of human sRAGE using insect cell culture. In an embodiment, the method comprises (a) subcloning a nucleotide sequence encoding RAGE or a fragment thereof into a virus; (b) preparing a high-titer stock of recombinant virus; and (c) infecting host cells with the high-titer recombinant virus under conditions such that pre-determined levels of RAGE or a fragment thereof is produced, wherein said pre-determined levels of RAGE are at least 25 mg recombinant protein per liter of culture. The invention comprises use of sRAGE prepared by the methods of the invention for treatment of AGE-related syndromes including complications associated with atherosclerosis, diabetes, kidney failure, systemic lupus nephritis or inflammatory lupus nephritis, amyloidoses, Alzheimer's disease, cancer, inflammation, and erectile dysfunction.

This application claims priority to U.S. Provisional Application Serial No. 60/273,418, filed Mar. 5, 2001. The disclosure of U.S. Provisional Application Serial No. 60/273,418 is hereby incorporated by reference in its entirety.

### FIELD OF THE INVENTION

The invention relates to methods for high level expression of recombinant forms of the Receptor for Advanced Glycated Endproducts (RAGE). More particularly, the present invention describes high level expression of sRAGE in insect cells.

### BACKGROUND

Incubation of proteins or lipids with aldose sugars results in nonenzymatic glycation and oxidation of amino groups on proteins to form Amadori adducts. Over time, the adducts undergo additional rearrangements, dehydrations, and cross-linking with other proteins to form complexes known as Advanced Glycosylation End Products (AGEs). Factors which promote formation of AGEs included delayed protein turnover (e.g. as in amyloidoses), accumulation of macromolecules having high lysine content, and high blood glucose levels (e.g. as in diabetes) (Hori et al., J. Biol. Chem. 270: 25752-761, (1995)). AGEs have been implicated in a variety of disorders including complications associated with diabetes and normal aging.

AGEs display specific and saturable binding to cell surface receptors on endothelial cells of the microvasculature, monocytes, macrophages, smooth muscle cells, mesengial cells, and neurons. The Receptor for Advanced Glycated Endproducts (RAGE) is a member of the immunoglobulin super family of cell surface molecules. The extracellular (N-terminal) domain of RAGE includes three immunoglobulin-type regions: one variable (V) type domain followed by two constant (C) type domains (Neeper et al., J. Biol. Chem., 267:14998-15004 (1992); Schmidt et al., Circ. (Suppl.) 96#194 (1997)). A single transmembrane spanning domain and a short, highly charged cytosolic tail follow the extracellular domain. The N-terminal, extracellular domain can be isolated by proteolysis of RAGE to generate soluble RAGE (sRAGE) which includes the V domain and both C domains (Neeper et al., J. Biol. Chem., 267:14998-15004 (1992); Schmidt et al., Circ. (Suppl.) 96#194 (1997)).

RAGE is expressed in most tissues, and in particular, is found in cortical neurons during embryogenesis (Hori et al., J. Biol. Chem., 270:25752-761 (1995)). Increased levels of RAGE are also found in aging tissues (Schleicher et al., J. Clin. Invest., 99 (3): 457-468 (1997)), and the diabetic retina, vasculature and kidney (Schmidt et al., Nature Med., 1:1002-1004 (1995)). Activation of RAGE in different tissues and organs leads to a number of pathophysiological consequences. RAGE has been implicated in a variety of conditions including: acute and chronic inflammation (Hofmann et al, Cell, 97:889-901 (1999)) the development of diabetic late complications such as increased vascular permeability (Wautier et al., J. Clin. Invest., 97:238-243 (1995), nephropathy (Teillet et al., J. Am. Soc. Nephrol., 11:1488-1497 (2000), atherosclerosis (Vlassara et. al., The Finnish Medical Society DUODECIM, Ann. Med., 28:419-426 (1996), and retinopathy (Hammes et al., Diabetologia, 42:603-607 (1999)). RAGE has also been implicated in Alzheimer's disease (Yan et al., Nature, 382: 685-691 (1996); erectile dysfunction; and in tumor invasion and metastasis (Taguchi et al., Nature, 405: 354-357 (2000)).

In addition to AGEs, other compounds can bind to, and modulate RAGE. In normal development, RAGE interacts with amphoterin, a polypeptide which mediates neurite outgrowth in cultured embryonic neurons (Hori et al., 1995). RAGE has also been shown to interact with β-amyloid (Yan et al., Nature 389:589-595 (1997); Yan et al., Nature 382:685-691 (1996); Yan et al., Proc. Natl. Acad. Sci. (USA) 94:5296-5301 (1997)).

It has been shown that sRAGE can be used to inhibit binding of AGEs and other ligands to RAGE (Schmidt et al., J. Biol. Chem., 267:14987-14997 (1992); Yan et al., Proc. Natl. Acad. Sci. (USA) 94:5296-5301 (1997); Park et al., Nature Med., 4:1025-1031 (1998); Kislinger et al., J. Biol. Chem., 274:31740-31749 (1999)). By interfering with binding of ligands to RAGE, sRAGE can be used to ameliorate the effects of excess AGEs. Thus, sRAGE can be used to treat disease symptoms which result from excess activation of RAGE, as for example, in diabetes, inflammation, accelerated atherosclerosis, and Alzheimer's disease.

There is, therefore, a need for the development of systems which express RAGE and physiologically active subfragments such as sRAGE. The recombinant protein should be processed by the host cell so that the final protein product comprises therapeutically active human RAGE, or a fragment thereof. In addition, the recombinant should be expressed in high yield, thereby allowing purification and distribution on a commercial scale.

### SUMMARY

In one aspect, the invention comprises methods for high level expression of recombinant forms of the Receptor for Advanced Glycated Endproducts (RAGE) or fragments thereof comprising subcloning a nucleotide sequence encoding RAGE or a fragment thereof into a baculovirus or other viral expression vector; preparing a high titer virus stock; and infecting host cells under conditions such that a predetermined level of RAGE or a fragment thereof is produced, wherein a pre-determined level of RAGE is at least 25 mg recombinant protein per liter of culture.

In one aspect, the invention comprises methods for high level expression of recombinant human Receptor for Advanced Glycated Endproducts (RAGE) comprising:

(a) preparing recombinant virus comprising a nucleotide sequence encoding RAGE or a fragment thereof subcloned into the Autographa californica nuclear polyhedrosis virus; (b) preparing a high titer stock of the recombinant virus; (c) initiating cultures of insect cells at an initial density of less than 0.5×10⁶ cells per ml; (d) growing the insect cells until the cell density comprises 1 to 20×10⁶ cells per ml; (e) adding virus from step (b) at a MOI of less than 30 to the insect cells from step (d); and (f) incubating the infected culture at about 26-28° C. for a time period sufficient to produce a predetermined amount of RAGE protein.

In another aspect, the invention comprises a method for high level expression of recombinant human Receptor for Advanced Glycated Endproducts (RAGE) comprising:

(a) preparing recombinant virus comprising a nucleotide sequence encoding RAGE or a fragment thereof subcloned into the Autographa californica nuclear polyhedrosis virus; (b) infecting insect cells at a multiplicity of infection (MOI) of about 0.1 to 0.2; (c) incubating the insect cell culture at a temperature ranging from about 26-28° C. for 3-7 days to prepare high titer virus stock; (d) titering the virus to determine MOI; (e) initiating cultures of insect cells at an initial density of about 2.5×10⁵ cells per ml; (f) growing the insect cells until the cell density comprises 1.5 to 2.5×10⁶ cells per ml; (g) adding virus (from step (d)) at a MOI of 0.1 to 10 to the insect cells; and (h) incubating the infected culture at about 26-28° C. for a time period sufficient to produce a predetermined amount of RAGE protein.

In another aspect, the invention comprises using recombinant RAGE compounds made by the method of the invention for treating human disease.

The foregoing focuses on the more important features of the invention in order that the detailed description which follows may be better understood and in order that the present contribution to the art may be better appreciated. There are, of course, additional features of the invention which will be described hereinafter and which will form the specification appended hereto. It is to be understood that the invention is not limited in its application to the details set forth in the following description and drawings. The invention is capable of other embodiments and of being practiced or carried out in various ways.

From the foregoing summary, it is apparent that an object of the present invention is to provide a system which allows the expression of RAGE, sRAGE and physiologically active fragments of RAGE such as the V-domain of RAGE. These, together with other objects of the present invention, along with the various features of novelty which characterize the invention, are pointed out with particularity in the description and drawings herein.

### BRIEF DESCRIPTION OF THE FIGURES

Various features, aspects and advantages of the present invention will become more apparent with reference to the following description and accompanying drawings.

FIG. 1 shows a schematic representation of an embodiment of the method of the present invention.

FIG. 2 shows (A) SEQ ID NO: 1, the nucleotide sequence (in the 5′ to 3′ direction) of human RAGE as reported in GenBank/EMBL database accession no. XM004205; (B) SEQ ID NO: 2, the nucleotide sequence (in the 5′ to 3′ direction) of human sRAGE subcloned into the pBacPAK baculovirus vector and SEQ ID NO: 3, the amino acid sequence of sRAGE subcloned into the pBacPAK baculovirus vector; and (C) SEQ ID NO: 4, the nucleotide sequence (in the 5′ to 3′ direction) of the V-domain of human RAGE, each in accordance with preferred embodiments of the present invention.

FIG. 3 shows a diagram of human sRAGE insert subcloned into pBacPAK8 and the sequencing strategy used to verify the sequence in accordance with an embodiment of the present invention.

FIG. 4 shows an SDS PAGE gel of cell pellets (P) and supernatants (S) for insect cell recombinants generated in accordance with an embodiment of the present invention from a time course of infection using an MOI of 0.1.

### DETAILED DESCRIPTION

Thus, the present invention relates to the production of recombinant RAGE, or fragments of RAGE, such as sRAGE or the V-domain of RAGE. These recombinant preparations may be used for further characterization of the physiological pathways by which RAGE mediates the response to AGEs, or as therapeutics, for treatment of diseases caused by increase levels of circulating AGEs.

In one aspect, the invention comprises methods for high level expression of recombinant forms of the Receptor for Advanced Glycated Endproducts (RAGE) or fragments thereof comprising subcloning a nucleotide sequence encoding RAGE or a fragment thereof into a virus; preparing a high-titer stock of recombinant virus; and infecting host cells with the high-titer recombinant virus under conditions such that pre-determined levels of RAGE or a fragment thereof is produced, wherein a pre-determined level of RAGE comprises at least 25 mg recombinant protein per liter of culture.

Preferably, the method further comprises a yield of recombinant protein comprising more than 50 mg per liter of culture. More preferably, the method comprises a yield of recombinant protein comprising more than 100 mg per liter of culture. Even more preferably, the method comprises a yield of recombinant protein comprising more than 250 mg per liter of culture.

Preferably the virus comprises the Autographa californica nuclear polyhedrosis virus. More preferably, the virus comprises BacPAK6 or similar systems. Also, preferably, the recombinant RAGE protein or fragment thereof is purified from the insect media using Sepharose.

In an embodiment, the host cells comprise insect cells such as Sf9 or Sf21 cells. Preferably, the step of preparing the high-titer recombinant virus stock comprises infecting insect cells at a multiplicity of infection (MOI) of less than 1, and incubating the insect cell culture at a temperature of about 26-28° C. for 3-7 days to prepare high titer virus stock. In an embodiment, the innoculum used to prepare the high titer stock comprises an MOI of 0.01 to 1.0. More preferably, the initial MOI comprises 0.05 to 0.5. Even more preferably, the initial MOI comprises 0.1 to 0.2.

In an embodiment, the nucleic acid sequence encoding RAGE is the nucleic acid sequence SEQ ID NO: 1, or a sequence substantially homologous thereto. In an embodiment, the fragment of RAGE is the soluble, extracellular portion of RAGE (sRAGE), as defined by the nucleic acid sequence SEQ ID NO: 2, or a sequence substantially homologous thereto. In an embodiment, the fragment of RAGE is the V-domain of RAGE, as defined by the nucleic acid sequence SEQ ID NO: 4, or a sequence substantially homologous thereto.

Preferably, infecting host cells under conditions such that high levels of RAGE or a fragment thereof is produced comprises initiating cultures of insect cells at a low density; growing the insect cells to a preset final density; and adding the high titer virus at a MOI of less than 30; and incubating infected cells under conditions such that a predetermined level of RAGE or a fragment thereof is produced.

Preferably, the step of infecting cells at a low density comprises cells having an initial density of less than 0.5×10⁶ cells per ml. Also preferably, cells are grown from an initial density of less than 0.5×10⁶ cells per ml to a final density comprising 1 to 20×10⁶ cells per ml. Also preferably, the cells are grown under conditions comprising a pre-set doubling time and viability. In an embodiment, the rate of cell growth is monitored. Preferably, the rate of cell growth comprises a doubling rate of 10-35 hours. More preferably, the rate of cell growth comprises a doubling rate of 15-30 hours. Even more preferably, the rate of cell growth comprises a doubling rate of 18-26 hours. Also more preferably, the doubling time comprises conditions such that cell viability is greater than 90%.

Preferably the conditions of incubating infected cells to produce predetermined levels of RAGE or a fragment thereof are determined by MOI time course experiments. Also preferably, the conditions of incubating infected cells to produce high levels of RAGE or a fragment thereof comprise visual inspection of the culture to ascertain the point at which cultures become cloudy.

In one aspect, the invention comprises method for high level expression of recombinant human Receptor for Advanced Glycated Endproducts (RAGE) comprising: (a) preparing recombinant virus comprising a nucleic acid sequence encoding RAGE or a fragment thereof subcloned into the Autographa californica nuclear polyhedrosis virus; (b) preparing a high-titer stock of the recombinant virus; (c) initiating cultures of insect cells at an initial density of less than 0.5×10⁶ cells per ml; (d) growing the insect cells until the cell density comprises 1 to 20×10⁶ cells per ml; (e) adding virus from step (b) at a MOI of less than 30 to the insect cells from step (d) for large scale espression; and (f) incubating the infected culture at about 26-28° C. under conditions such that a predetermined level of RAGE or a fragment thereof is produced. Preferably, the recombinant RAGE or fragment thereof is purified from the insect media using Sepharose.

Preferably, the method comprises a yield of recombinant protein comprising at least 25 mg per liter of culture. More preferably, the method comprises a yield of recombinant protein comprising more than 50 mg per liter of culture. More preferably, the method comprises a yield of recombinant protein comprising more than 100 mg per liter of culture. Even more preferably, the method comprises a yield of recombinant protein comprising more than 250 mg per liter of culture.

In an embodiment, the nucleic acid sequence encoding RAGE is the nucleic acid sequence SEQ ID NO: 1, or a sequence substantially homologous thereto. In an embodiment, the fragment of RAGE is the soluble, extracellular portion of RAGE (sRAGE), as defined by the nucleic acid sequence SEQ ID NO: 2, or a sequence substantially homologous thereto. In an embodiment, the fragment of RAGE is the V-domain of RAGE, as defined by the nucleic acid sequence SEQ ID NO: 4, or a sequence substantially homologous thereto.

Preferably, the step of preparing the recombinant virus stock comprises infecting insect cells at a multiplicity of infection (MOI) of less than 1, and incubating the insect cell culture at a temperature ranging from about 26-28° C. for 3-7 days to prepare a high titer virus stock. In an embodiment, the MOI used to prepare the high titer virus stock comprises 0.01 to 1.0. More preferably, the initial MOI comprises 0.05 to 0.5. Even more preferably, the initial MOI comprises 0.1 to 0.2.

Also preferably, the cells infected used for large scale expression are grown under conditions comprising a pre-set doubling time and viability. In an embodiment, the rate of cell growth is monitored. Preferably, the rate of cell growth comprises a doubling rate of 10-35 hours. More preferably, the rate of cell growth comprises a doubling rate of 15-30 hours. Even more preferably, the rate of cell growth comprises a doubling rate of 18-26 hours. Also more preferably, the doubling time comprises conditions such that cell viability is greater than 90%.

Preferably, the culture used to prepare the high titer virus is grown for 3-7 days. More preferably, the culture used to prepare the high titer virus is grown for 4-6 days. Even more preferably, the culture used to prepare the high titer virus is grown for about 5 days.

Preferably the conditions of incubating infected cells (step f) to produce predetermined levels of RAGE or a fragment thereof are determined by MOI time course experiments. Also preferably, the conditions of incubating infected cells to produce high levels of RAGE or a fragment thereof comprise visual inspection of the culture to ascertain the point at which cultures become cloudy.

In yet another aspect, the invention comprises a method for high level expression of recombinant human Receptor for Advanced Glycated Endproducts (RAGE) comprising: (a) preparing recombinant virus comprising a nucleotide sequence encoding RAGE or a fragment thereof, subcloned into the Autographa californica nuclear polyhedrosis virus; (b) infecting insect cells at a multiplicity of infection (MOI) of about 0.1 to 0.2; (c) incubating the insect cell culture at a temperature ranging from 26-28° C. for 3-7 days to prepare high titer virus stock; (d) titering the virus to determine MOI; (e) initiating cultures of insect cells at an initial density of about 2.5×10⁵ cells per ml; (f) growing the insect cells such that the growth rate comprises a doubling time of about 18-26 hours and the cells comprise a viability of greater than 90% until the cell density comprises 1.5 to 2.5×10⁶ cells per ml; (g) adding virus (from step (d)) at a MOI of 0.1 to 10 to the insect cells; and (h) incubating the infected culture at about 26-28° C. for a predetermined time or until cloudy. Preferably, the recombinant RAGE protein or fragment thereof is purified from the insect media using Sepharose.

Preferably, the method comprises a yield of recombinant protein comprising more than 25 mg per liter of culture. Preferably, the method comprises a yield of recombinant protein comprising more than 50 mg per liter of culture. More preferably, the method comprises a yield of recombinant protein comprising more than 100 mg per liter of culture. Even more preferably, the method comprises a yield of recombinant protein comprising more than 250 mg per liter of culture.

In an embodiment, the nucleic acid sequence encoding RAGE is the nucleic acid sequence SEQ ID NO: 1, or a sequence substantially homologous thereto. In an embodiment, the fragment of RAGE is the soluble, extracellular portion of RAGE (sRAGE), as defined by the nucleic acid sequence SEQ ID NO: 2, or a sequence substantially homologous thereto. In an embodiment, the fragment of RAGE is the V-domain of RAGE, as defined by the nucleic acid sequence SEQ ID NO: 4, or a sequence substantially homologous thereto.

In another aspect, the present invention comprises insect cells producing recombinant RAGE or a fragment thereof according to the methods of the present invention.

In yet another aspect, the present invention comprises recombinant RAGE produced by the methods of the invention.

In an embodiment, the present invention also comprises a method of treating human disease comprising administering recombinant RAGE polypeptide made by the methods of the invention in a pharmaceutically acceptable carrier. Preferably, the human diseases treated using the recombinant RAGE polypeptide comprise atherosclerosis, diabetes, symptoms of diabetes late complications, amyloidosis, Alzheimer's Disease, cancer, inflammation, kidney failure, systemic lupus nephritis, inflammatory lupus nepritis, or erectile dysfunction.

In another embodiment, the present invention comprises a method for inhibiting the interaction of an advanced glycosylation end products (AGE) with RAGE in a subject, comprising administering to the subject a therapeutically effective amount of recombinant RAGE polypeptide made by the methods of the present invention. In an embodiment, the recombinant RAGE is administered as a therapeutically effective amount of recombinant sRAGE with an appropriate pharmaceutical so as to prevent or ameliorate disease associated with increased levels of advanced glycosylation end products (AGEs). Also, the disease associated with increased levels of AGEs preferably comprises accelerated atherosclerosis, diabetes, Alzheimer's Disease, inflammation, systemic lupus nephritis, inflammatory lupus nephritis, cancer, or erectile dysfunction.

Preferably, an effective amount of sRAGE ranges from about 1 ng/kg body weight to 100 mg/kg body weight. More preferably, an effective amount of sRAGE ranges from about 1 μg/kg body weight to 50 mg/kg body weight. Even more preferably, an effective amount of sRAGE ranges from about 10 μg/kg body weight to 10 mg/kg body weight.

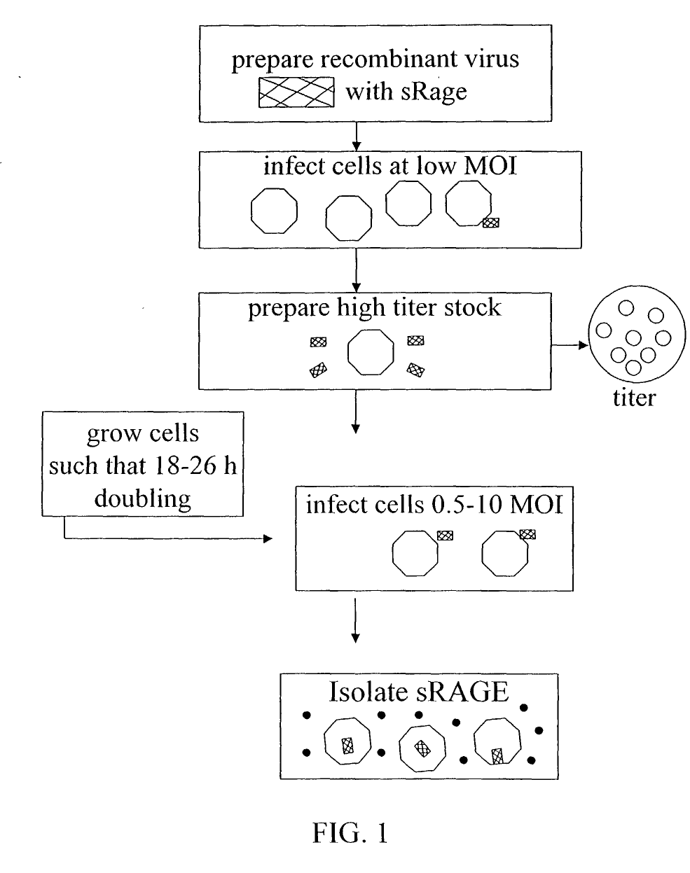

Thus, the invention comprises the use of baculovirus to generate high levels of mammalian RAGE protein using cultures of insect cells. Referring now to FIG. 1, it can be seen that the method comprises: (1) preparing a recombinant virus with a fragment of RAGE such as sRAGE; (2) infecting host cells at a low MOI to prepare a virus stock which is optimized with respect to MOI; (3) performing an MOI time course and titering the stock to determine the isolate comprising optimum MOI; (4) starting a separate culture of host cells comprising a doubling time of about 18-26 hours with >90% viability; (5) infecting the growing cells with the optimized high-titer stock using an MOI of 0.5 to 10 and allowing the cells to incubate under conditions such that large amounts of recombinant sRAGE are made; and (6) purifying sRAGE from the media.

Insect cells are increasingly used for production of recombinant proteins. In most cases, posttranslational processing of eukaryotic proteins in insect cells is similar to protein processing in mammalian cells. For example, a baculovirus commonly used to express foreign proteins is Autographa californica nuclear polyhedrosis virus (AcMNPV) (see e.g. Luckow, BioTechnology 6:47-55 (1991)). The baculovirus AcMNPV begins replication at about 6 hours post-infection (pi). At about 20 to 48 hours, transcription of all the viral genes except for the polyhedrin and p10 genes ceases. The polyhedrin protein, while essential for propagation of the virus in its natural habitat, is not required for propagation of the virus in cell culture, and thus, can be replaced with a foreign sequence. Replacement of polyhedrin gene sequences with an inserted foreign sequence enables expression of the inserted gene by the polyhedrin promoter.

Because the AcMNPV genome is fairly large, recombinant baculovirus expression vectors may employ recombination between a transfer vector comprising insert DNA and the viral genome. For example, in the pBacPAK system (Clontech, Palo Alto, Calif.) a target gene is cloned into a polyhedrin locus which is contained in a relatively small (<10 kb) transfer vector. The polyhedrin locus in the transfer vector has the coding sequence deleted and replaced with a multiple cloning site (MCS) for insertion of a target gene between the polyhedrin promoter and polyadenylation signals. In a second step, the transfer vector (which is unable to replicate on its own in insect cells) and a viral genomic DNA are co-transfected into insect cells. Double recombination between viral sequences in the transfer vector and the corresponding sequences in the viral DNA transfers the target gene to the viral genome to generate a viral expression vector.

AcMNPV-based expression systems generally produce adequate quantities of proteins localized to the nucleus or cytoplasm (U.S. Pat. No. 5,179,007). However, proteins processed by the endoplasmic reticulum such as cell surface receptors (Chazenbalk et al., J. Biol Chem., 270: 1543-1549 (1995)), antibodies (Hsu et al., Prot. Expr. Purif, 5: 595-603 (1994)), and secreted vaccine components (Li et al., Virology, 204: 266-278 (1994)), are expressed at lower levels (Jarvis, Insect Cell Culture Engineering, Macel Dekker, Inc., New York, N.Y. (1993)).

Others have tried to address the low expression levels seen for some proteins. For example, WO 98/44141 describes the use of insect shuttle vectors for stably transforming insect cells that employ: (1) a prokaryotic origin of replication; (2) an insect promoter having homology to, and capable of functioning as an immediate early baculovirus promoter; (3) a prokaryotic promoter sequence; (4) a resistance gene to a biomycin/phleomycin-type antibiotic under the control of the insect promoter and prokaryotic promoters; and (5) in some embodiments, a transposon. Also, WO 99/31257 describes the use of infection with baculovirus comprising a cell density of 1×10⁵ to 1×10⁶ cells/ml and a low viral innoculum (i.e. <0.01 MOI) for the generation of recombinant protein at levels comprising 100 μg/ml. Still, WO 99/31257 describes methods for production of pestivirus E2 or E^{rns} protein, viral envelop proteins which are considerably different structure than RAGE and fragments thereof.

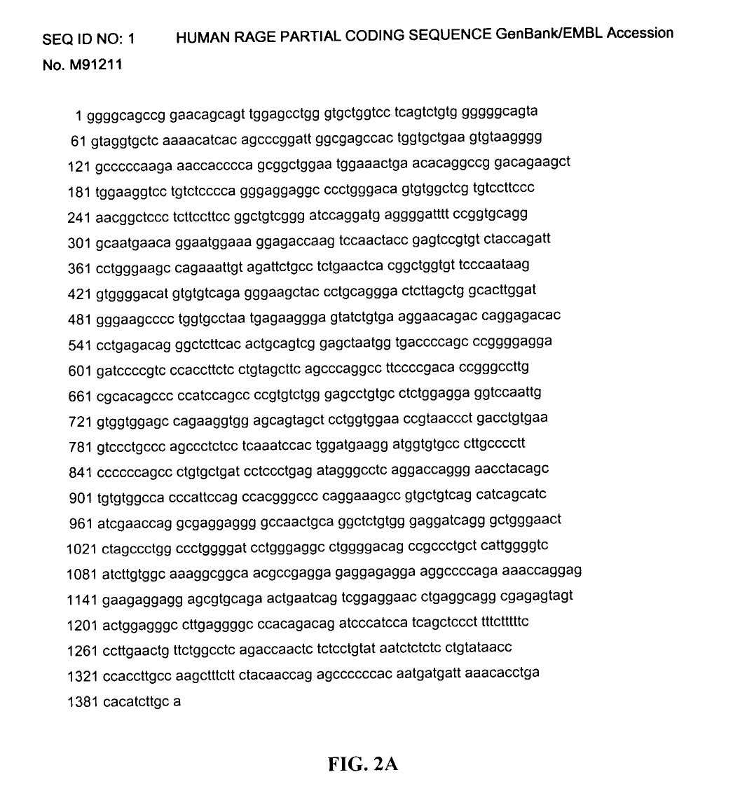

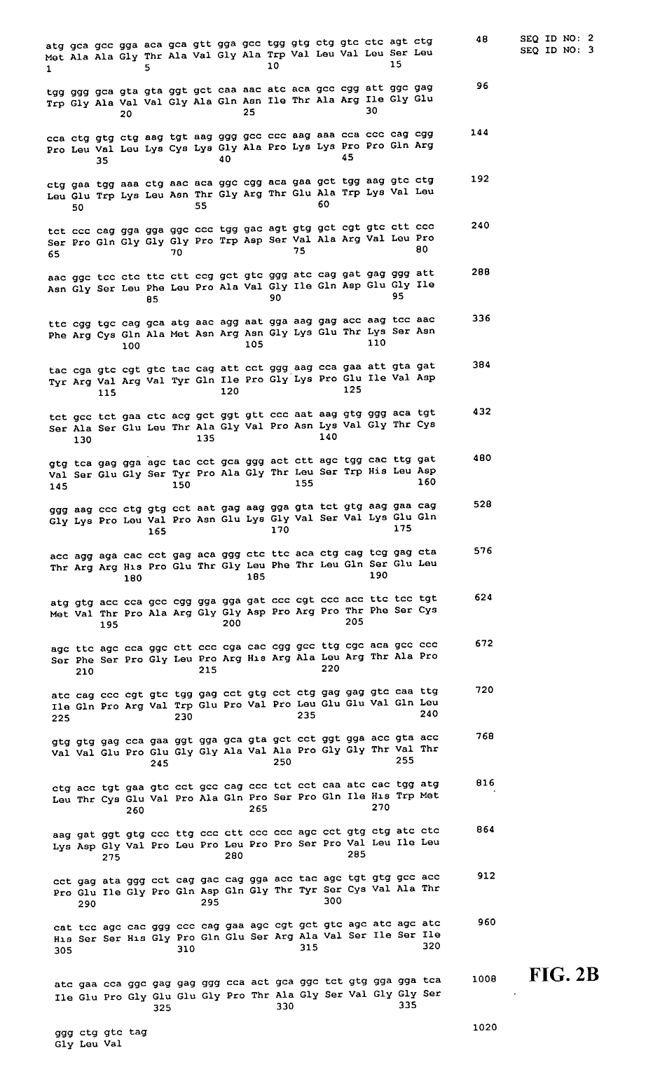

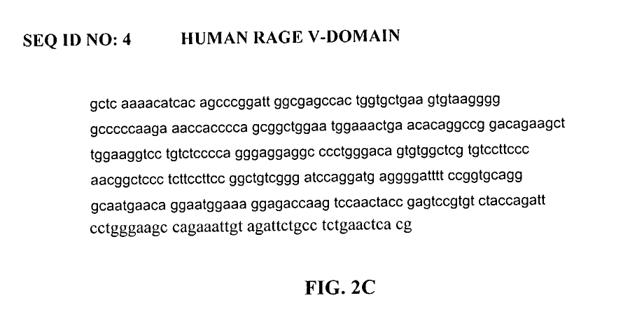

As used herein, RAGE encompasses the nucleic acid sequence shown in FIG. 2A (SEQ ID NO: 1) or fragment thereof (Neeper et al., (1992)). The binding domain of RAGE comprises that region of the gene which encodes a peptide which is able to bind ligands with physiological specificity. A fragment of RAGE is a sequence from the gene which is at least 15 nucleic acids in length, and more preferably greater than 150 nucleic acids in length, but is substantially less than the full sequence. Thus, in an embodiment, the fragment of RAGE comprises sRAGE (SEQ ID NO: 2; FIG. 2B), wherein sRAGE comprises the nucleic acid sequence that encodes the region of RAGE protein which is extracellular (Park et al., Nature Med., 4:1025-1031 (1998)). In another embodiment, the fragment of RAGE comprises the nucleic acid sequence encoding the V domain of RAGE (SEQ ID NO: 4; FIG. 2C) (Neeper et al., (1992)).

It will be understood that the invention comprises nucleic acid sequences substantially homologous to RAGE and fragments thereof. Substantial homology in the nucleic acid context means that the sequences of interest, or their complementary strands are the same when aligned, with in some cases deletions and insertions, in at least 60% of the nucleotides, and more preferably at least about 75% of the nucleotides, and more preferably at least about 90% of the nucleotides, and even more preferably at least about 95% of the nucleotides.

It will also be understood that the invention comprises the use of nucleic acid recombinants that generate proteins with substantial homology to the protein sequences of RAGE, sRAGE and the V-domain of RAGE. The terms “substantially homologous” when referring to polypeptides refer to at least two amino acid sequences which when optimally aligned, are at least 75% homologous, preferably at least about 85% homologous, more preferably at least about 90% homologous, and still more preferably 95% homologous. Optimal alignment of sequences for aligning a comparison may be conducted using the algorithms standard in the art (e.g. Smith and Waterman, Adv. Appl. Math. 2:482 (1981); Needleman and Wunsch, J. Mol. Biol. 48:443 (1970); Pearson and Lipman, Proc. Natl. Acad. Sci., USA, 85:2444 (1988) or by computerized versions of these algorithms (Wisconsin Genetics Software Package Release 7.0, Genetics Computer Group, 575 Science Drive, Madison, Wis.).

In an embodiment, recombinant virus is made by co-transfecting sRAGE which has been subcloned into a pBacPAK8 vector with BacPAK6 virus (Clontech, Palo Alto, Calif.) into insect host cells. More preferably, the human sRAGE is subcloned into the EcoRI and NotI sites of the multiple cloning site of the baculovirus vector pBacPAK8 to generate a sRAGE-pBacPAK8 recombinant. BacPAK6 has an essential gene adjacent to the polyhedrin locus that provides selection of recombinant viruses (Kitts et al., Biotechniques, 14:810-817 (1993); Clontech Manual, available at www.clontech.com). The BacPAK6 virus has been engineered such that digestion of the viral DNA with the restriction endonuclease Bsu36 I, releases two fragments, one (ORF1629) which is essential for viral replication; and a second larger fragment, which will not be viable alone, but must recombine back with the smaller fragment to be infective (Possee et al., Virol., 185:229-241 (1991)). The BacPAK system transfer vector is designed to contain the ORF1629 fragment linked to the polyhedrin locus and a multiple cloning site (MCS) into which foreign DNA is inserted. Thus, double recombination between the transfer vector and the large viral fragment generates a large circular viral DNA comprising all the genes necessary for viral replication and the inserted gene.

Preferably, recombinant virus is made by co-transfecting the sRAGE-pBacPAK8 recombinant with BacPAK viral DNA into insect host cells. More preferably, the insect cells comprise Spodoptera frugiperda such as sf9 and sf21 cells and the like. In an embodiment, the cells comprise Trichoplusia ni such as High Five, Tn-368 and the like. For example, the IPLB-Sf21 cell line (Sf21) is originally developed from the fall army worm, Spodoptera frugiperda (Vaughn et al., In Vitro, 13:213-217 (1977)). These cells grow well at temperatures ranging from about 22° C. to 30° C. and do not require CO₂. At optimum growth temperature (27-28° C.), doubling time is generally about 20-24 hr. Cells may be cultured as monolayers or as liquid cultures. Preferably, cells are not passaged indefinitely, but are replaced at regular intervals with fresh cells.

Generally media comprise TNM-FH (Clontech, Palo Alto, Calif.), BML-TC/10, IPL-41, SF900II (LTI), Excel 420 (JRH Biosci), Insect Xpress (BioWhittaker), and the like. Generally formulations comprise TC Yeastolate, pluorinic F68, lipids, and at least one or two protein hydrolysates, such as primatone.

Preferably, the method further comprises titering the recombinant virus to determine an optimum multiplicity of infection (MOI), wherein MOI is defined as the number of viral particles in the innoculum per insect cell (or any host cell) in the culture. More preferably, the initial multiplicity of infection MOI is determined by a plaque assay, using procedures known in the art. The initial MOI can influence both the fraction of infected cells and the number of polyhedra per cell at the end of infection. At a low MOI (e.g. less than 5) infection will generally not be synchronous, and cells will be composed of non-infected and infected cells at different points in the cell cycle. Still, selecting a low MOI reduces the amount of viral stock which must be produced and minimizes the risk of generation of defective interfering particles.

In an embodiment, cultures are propagated by seeding a volume of complete (non-selective) medium with cells to give a starting density of 1-10×10⁵ cells/ml and the culture incubated at 27° C. with shaking at about 50-100 rpm so that cells are kept in supension. Cells density is then monitored daily until the culture reaches 1-5×10⁶ cells/ml (about 4 days) and cell viability monitored (e.g. by staining with trypan blue). The cells are then used to seed a fresh spinner/shaker flask at a density of 1-2.5×10⁵ cells/ml.

In an embodiment, co-infection with virus DNA (BacPAK6) and the recombinant transfer plasmid is performed by transfecting sf9 insect cells with pBacPAK8 comprising sRAGE and BacPAK6 viral DNA using procedures well known in the art (e.g. Clontech, Palo Alto, Calif.). Preferably, supernatant from the transfection is harvested and the MOI assayed by plaque assay or other methods known in the art. Also preferably, the recombinant virus is then used at an MOI comprising less than 1 to infect insect cells. More preferably, recombinant virus is then used at an MOI comprising 0.01 to 1 to infect insect cells. More preferably, recombinant virus is then used at an MOI comprising 0.05 to 0.5 to infect insect cells. Even more preferably, recombinant virus is then used at an MOI comprising 0.1 to 0.2 to infect insect cells.

In an embodiment, cells infected with recombinant virus are grown for 3-7 days. Preferably, cells infected with recombinant virus are grown for 4-6 days. More preferably, cells infected with recombinant virus are grown for 5 days.

Preferably, supernatant from the infected cells is then harvested and test expressed at 0.1-10% V/V. More preferably, supernatant from the infected cells is harvested and test expressed at 0.5 to 2% V/V. Even more preferably, supernatant from the infected cells is harvested and test expressed at 1% V/V.

In an embodiment, sf9 or sf21 cells are used for large scale cell infection. Preferably, cells are used to inoculate medium at an initial cell density of no more than 0.5×10⁶ cells per ml. More preferably, cells are used to inoculate medium at an initial cell density of 0.5×10⁵ to 5×10⁵ cells per ml. Even more preferably, cells are used to inoculate medium at an initial cell density of about 2.5×10⁵ cells per ml.

In an embodiment, the rate of cell growth is monitored. Preferably, the rate of cell growth comprises a doubling rate of 10-35 hours. More preferably, the rate of cell growth comprises a doubling rate of 15-30 hours. Even more preferably, the rate of cell growth comprises a doubling rate of 18-26 hours. Also more preferably, the doubling time comprises conditions such that cell viability is greater than 90%.

In an embodiment, the recombinant virus is added to the insect cells when the cell density has increased about 10-fold of the original density. Preferably, the cell density when the recombinant virus is added comprises 1×10⁵ to 1×10⁷ cells/ml. More preferably, the cell density when the recombinant virus is added comprises 1 to 20×10⁶ cells/ml. Even more preferably, the cell density when the recombinant virus is added comprises 1.5-2.5×10⁶ cells/ml.

In an embodiment, the virus is added to the liquid culture suspension at a known MOI. Preferably the MOI comprises less than 30. More preferably, the MOI comprises 0.1 to 20. Even more preferably, the MOI comprises 0.1-10.

In an embodiment, the culture is incubated for an extended period after infection with the virus. In an embodiment the time of incubation is determined by a time course experiment using aliquots of the cells and virus comprising the large scale preparation. Preferably, the time of incubation comprises 24-96 hours. More preferably, the time of incubation comprises 36-80 hours. Even more preferably, the time of incubation comprises 48-72 hours.

In an embodiment, cells are grown on solid support. In an alternate embodiment, the cells are grown in suspension. Preferably, cells comprising the recombinant RAGE, or fragment thereof, are grown in a culture vessel with sufficient volume to contain up to 10 liters of growth medium. More preferably, cells comprising the recombinant RAGE, or fragment thereof, are grown in a culture vessel with sufficient volume to contain up to 50 liters of growth medium. More preferably, cells comprising the recombinant RAGE, or fragment thereof, are grown in a culture vessel with sufficient volume to contain up to 250 liters of growth medium. Even more preferably, cells comprising the recombinant RAGE, or fragment thereof, are grown in a culture with sufficient volume to contain up to 2,000 liters of growth medium.

An advantage of the method is the reproducibly high yields of recombinant RAGE expressed from the host cells. In an embodiment, the insect cells are grown in suspension in a culture vessel, such as a fermentor, which can be moderately stirred. Preferably, the method comprises a yield of recombinant protein comprising more than 25 mg per liter of culture. More preferably, the method comprises a yield of recombinant protein comprising more than 50 mg per liter of culture. More preferably, the method comprises a yield of recombinant protein comprising more than 100 mg per liter of culture. Even more preferably, the method comprises a yield of recombinant protein comprising more than 250 mg per liter of culture.

In an embodiment, expressed sRAGE is purified from the culture medium by absorption to SP-Sepharose equilibrated with 50 mM sodium phosphate, pH 5.6, and step-wise elution using sodium phosphate buffer, pH 5.6, and increasing salt. Preferably, a majority of the recombinant sRAGE is eluted in 50 mM sodium phosphate 1M NaCl, pH 5.6, and then de-salted by dialysis against 50 mM sodium phosphate, 150 mM NaCl, pH 6.5.

In one aspect, the invention comprises using compounds made by the methods of the invention for treating human disease. In an embodiment, the compound made by the methods of the invention is used to treat symptoms of diabetes and symptoms of diabetic late complications. In another embodiment, the compound made by the methods of the invention is used to treat amyloidosis. In another embodiment, the compound made by the methods of the invention is used to treat Alzheimer's disease. In yet another embodiment, the compound made by the methods of the invention may be used to treat cancer. In another embodiment, the compound made by the methods of the invention is used to treat inflammation. In yet another embodiment, the compound made by the methods of the invention is used to treat kidney failure. In yet another embodiment, the compound made by the methods of the invention is used to treat systemic lupus nephritis or inflammatory lupus nephritis. In yet another embodiment, the compound made by the method of the invention is used to treat erectile dysfunction.

Preferably, the recombinant RAGE, or a fragment thereof, produced by the methods of the invention is used to treat symptoms of diabetes. It has been shown that nonenzymatic glycoxidation of macromolecules ultimately resulting in the formation of advanced glycation endproducts (AGEs) is enhanced at sites of inflammation, in renal failure, in the presence of hyperglycemia and other conditions associated with systemic or local oxidant stress (Dyer et al., J. Clin. Invest., 91:2463-2469 (1993); Reddy et al., Biochem., 34:10872-10878(1995); Dyer et al., J. Biol. Chem., 266:11654-11660(1991); Degenhardt et al., Cell Mol. Biol., 44:1139-1145 (1998)). Accumulation of AGEs in the vasculature can occur focally, as in the joint amyloid composed of AGE-β₂-microglobulin found in patients with dialysis-related amyloidosis (Miyata et al., J. Clin. Invest., 92:1243-1252 (1993); Miyata et al., J. Clin. Invest., 98:1088-1094 (1996)), or generally, as exemplified by the vasculature and tissues of patients with diabetes (Schmidt et al., Nature Med., 1:1002-1004 (1995)). The progressive accumulation of AGEs over time in patients with diabetes suggests that endogenous clearance mechanisms are not able to function effectively at sites of AGE deposition. Such accumulated AGEs have the capacity to alter cellular properties by a number of mechanisms. Although RAGE is expressed at low levels in normal tissues and vasculature, in an environment where the receptor's ligands accumulate, it has been shown that RAGE becomes upregulated (Li et al., J. Biol. Chem., 272:16498-16506 (1997); Li et al., J. Biol. Chem., 273:30870-30878 (1998); Tanaka et al., J. Biol. Chem. 275:25781-25790(2000)). RAGE expression is increased in endothelium, smooth muscle cells and infiltrating mononuclear phagocytes in diabetic vasculature. Also, studies in cell culture have demonstrated that AGE-RAGE interaction caused changes in cellular properties important in vascular homeostasis.

Preferably, the recombinant RAGE or a fragment thereof produced by the methods of the invention is used to treat atherosclerosis. Thus, it has been shown that ischemic heart disease is particularly high in patients with diabetes (Robertson et al., Lab Invest., 18:538-551 (1968); Kannel et al, J. Am. Med. Assoc., 241:2035-2038 (1979); Kannel et al., Diab. Care, 2:120-126 (1979)). In addition, studies have shown that atherosclerosis in patients with diabetes is more accelerated and extensive than in patients not suffering from diabetes (see e.g. Waller et al., Am. J. Med., 69:498-506 (1980); Crall et al, Am. J. Med. 64:221-230 (1978); Hamby et al., Chest, 2:251-257 (1976); and Pyorala et al., Diab. Metab. Rev., 3:463-524 (1978)). Although the reasons for accelerated atherosclerosis in the setting of diabetes are many, it has been shown that reduction of AGEs can reduce plaque formation.

Preferably, the recombinant RAGE or a fragment thereof produced by the methods of the invention is used to treat amyloidoses and Alzheimer's disease. RAGE has been shown to function as a cell surface receptor which binds amyloid-β (Aβ) regardless of the composition of the subunits (amyloid-β peptide, amylin, serum amyloid A, prion-derived peptide) (Yan et al., Nature, 382:685-691 (1996); Yan et al., Nat. Med., 6:643-651 (2000)). Deposition of amyloid-β has been shown to result in enhanced expression of RAGE. For example, in the brains of patients with Alzheimer's disease (AD), RAGE expression increases in neurons and glia (Yan et al., Nature 382:685-691 (1996)). The consequences of Aβ interaction with RAGE appear to be quite different on neurons versus microglia. Whereas microglia become activated as a consequence of Aβ-RAGE interaction, as reflected by increased motility and expression of cytokines, early RAGE-mediated neuronal activation is superceded by cytotoxicity at later times. Further evidence of a role for RAGE in cellular interactions of Aβ concerns inhibition of Aβ-induced cerebral vasoconstriction and transfer of the peptide across the blood-brain barrier to brain parenchyma when the receptor was blocked (Kumar et al., Neurosci. Program, p141-#275.19 (2000)). Inhibition of RAGE-amyloid interaction has been shown to decrease expression of cellular RAGE and cell stress markers (as well as NF-kB activation), and diminish amyloid deposition (Yan et al., Nat. Med., 6:643-651 (2000)) suggesting a role for RAGE-amyloid interaction in both perturbation of cellular properties in an environment enriched for amyloid (even at early stages) as well as in amyloid accumulation.

Also preferably, the recombinant RAGE or a fragment thereof produced by the methods of the invention is used to treat cancer. For example, amphoterin is a high mobility group I nonhistone chromosomal DNA binding protein (Rauvala et al., J. Biol. Chem., 262:16625-16635 (1987); Parkikinen et al., J. Biol. Chem., 268:19726-19738 (1993)) which has been shown to interact with RAGE. It has been shown that amphoterin promotes neurite outgrowth, as well as serving as a surface for assembly of protease complexes in the fibrinolytic system (also known to contribute to cell mobility). In addition, a local tumor growth inhibitory effect of blocking RAGE has been observed in a primary tumor model (C6 glioma), the Lewis lung metastasis model (Taguchi et al., Nature 405:354-360 (2000)), and spontaneously arising papillomas in mice expressing the v-Ha-ras transgene (Leder et al., Proc. Natl. Acad. Sci., 87:9178-9182 (1990)).

Also preferably, the recombinant RAGE or a fragment thereof produced by the methods of the invention is used to treat inflammation. Also preferably, the compound identified by the methods of the invention is used to treat kidney failure. Also preferably, the compound identified by the methods of the invention is used to treat systemic lupus nephritis or inflammatory lupus nephritis. For example, the S100/calgranulins have been shown to comprise a family of closely related calcium-binding polypeptides characterized by two EF-hand regions linked by a connecting peptide (Schafer et al., TIBS, 21:134-140 (1996); Zimmer et al., Brain Res. Bull., 37:417-429 (1995); Rammes et al., J. Biol. Chem., 272:9496-9502 (1997); Lugering et al., Eur. J. Clin. Invest., 25:659-664 (1995)). Although they lack signal peptides, it has long been known that S100/calgranulins gain access to the extracellular space, especially at sites of chronic immune/inflammatory responses, as in cystic fibrosis and rheumatoid arthritis. RAGE is a receptor for many members of the S100/calgranulin family, mediating their proinflammatory effects on cells such as lymphocytes and mononuclear phagocytes. Also, studies on delayed-type hypersensitivity response, colitis in IL-10 null mice, collagen-induced arthritis, and experimental autoimmune encephalitis models suggest that RAGE-ligand interaction (presumably with S100/calgranulins) has a proximal role in the inflammatory cascade.

Also preferably, the recombinant RAGE or a fragment thereof produced by the methods of the invention is used to treat erectile dysfunction. Relaxation of the smooth muscle cells in the cavernosal arterioles and sinuses results in increased blood flow into the penis, raising corpus cavernosum pressure to culminate in penile erection. Nitric oxide is considered the principle stimulator of cavernosal smooth muscle relaxation (Chitaley et al, Nature Medicine, Jan;7(1):119-122 (2001)). RAGE activation produces oxidants (Yan et a.l, J. Biol. Chem., 269:9889-9887, 1994) via an NADH oxidase-like enzyme, therefore suppressing the circulation of nitric oxide. Potentially by inhibiting the activation of RAGE signaling pathways by decreasing the intracellular production of AGEs, generation of oxidants will be attenuated. Rage blockers may promote and facilitate penile erection by blocking the access of ligands to RAGE. The calcium-sensitizing Rho-kinase pathway may play a synergistic role in cavernosal vasoconstriction to maintain penile flaccidity. The antagonism of Rho-kinase results in increased corpus cavernosum pressure, initiating the erectile response independently of nitric oxide (Chitaley et al., 2001). One of the signaling mechanisms activated by RAGE involves the Rho-kinase family such as cdc42 and rac (Huttunen et al., J Biol Chem 274:19919-24 (1999)). Thus, inhibiting activation of Rho-kinases via suppression of RAGE signaling pathways will enhance and stimulate penile erection independently of nitric oxide.

In one aspect, the present invention also provides a method for inhibiting the interaction of an AGE with RAGE in a subject which comprises administering to the subject a therapeutically effective amount of the recombinant RAGE produced by the methods of the invention. For example, in an embodiment, the invention comprises administering to an individual a therapeutically effective amount of recombinant sRAGE produced by the method of the invention with an appropriate pharmaceutical so as to prevent or ameliorate accelerated atherosclerosis. In an embodiment, the invention comprises administering to an individual a therapeutically effective amount of recombinant sRAGE produced by the method of the invention with an appropriate pharmaceutical so as to prevent or ameliorate the symptoms of diabetes. In an embodiment, the invention comprises administering to an individual a therapeutically effective amount of recombinant sRAGE produced by the method of the invention with an appropriate pharmaceutical so as to prevent or ameliorate the symptoms of Alzheimer's disease. In an embodiment, the invention comprises administering to an individual a therapeutically effective amount of recombinant sRAGE produced by the method of the invention with an appropriate pharmaceutical so as to prevent or ameliorate inflammation. In an embodiment, the invention comprises administering to an individual a therapeutically effective amount of recombinant sRAGE produced by the method of the invention with an appropriate pharmaceutical so as to prevent or ameliorate cancer. In an embodiment, the invention comprises administering to an individual a therapeutically effective amount of recombinant sRAGE produced by the method of the invention with an appropriate pharmaceutical so as to prevent or ameliorate lupus. In yet another embodiment, the invention comprises administering to an individual a therapeutically effective amount of recombinant sRAGE produced by the method of the invention with an appropriate pharmaceutical so as to prevent or ameliorate erectile dysfunction.

A therapeutically effective amount is an amount which is capable of preventing interaction of AGE/RAGE in a subject. Accordingly, the amount will vary with the subject being treated. Administration of the compound may be hourly, daily, weekly, monthly, yearly or a single event. Preferably, the effective amount of the compound comprises from about 1 ng/kg body weight to about 100 mg/kg body weight. More preferably, the effective amount of the compound comprises from about 1 ug/kg body weight to about 50 mg/kg body weight. Even more preferably, the effective amount of the compound comprises from about 10 ug/kg body weight to about 10 mg/kg body weight. The actual effective amount will be established by dose/response assays using methods standard in the art (Johnson et al., Diabetes. 42: 1179, (1993)). Thus, as is known to those in the art, the effective amount will depend on bioavailability, bioactivity, and biodegradability of the compound.

In an embodiment, the subject is an animal. In an embodiment, the subject is a human. In an embodiment, the subject is suffering from an AGE-related disease such as diabetes, amyloidoses, renal failure, aging, or inflammation. In another embodiment, the subject comprises an individual with Alzheimer's disease. In an alternative embodiment, the subject comprises an individual with cancer. In yet another embodiment, the subject comprises an individual with systemic lupus erythmetosis, or inflammatory lupus nephritis.

In an embodiment, administration of the compound comprises intralesional, intraperitoneal, intramuscular, or intravenous injection. In an embodiment, administration of the compound comprises infusion or liposome-mediated delivery. In an embodiment, administration of the compound comprises topical application to the skin, nasal cavity, oral membranes or ocular tissue.

The pharmaceutically acceptable carriers of the invention comprise any of the standard pharmaceutically accepted carriers known in the art. In an embodiment, the carrier comprises a diluent. In an embodiment, the carrier comprises a liposome, a microcapsule, a polymer encapsulated cell, or a virus. For example, in one embodiment, the pharmaceutical carrier may be a liquid and the pharmaceutical composition in the form of a solution. In another embodiment, the pharmaceutically acceptable carrier is a solid and the composition is in the form of a powder or tablet. In a further embodiment, the pharmaceutical carrier is a gel or ointment and the composition is in the form of a suppository, cream, or liquid. Thus, the term pharmaceutically acceptable carrier encompasses, but is not limited to, any of the standard pharmaceutically accepted carriers, such as phosphate buffered saline solution, water, emulsions such as oil/water emulsions or trigyceride emulsion, various types of wetting agents, tablets, coated tablets and capsules.

For example, tablets or capsules may utilize pharmaceutically acceptable binding agents (e.g. polyvinylpyrrolidone, hydroxypropyl methylcellulose, starch); fillers (e.g. lactose, microcrystalline cellulose, calcium hydrogen phosphate); lubricants (e.g. magnesium stearate, silica or talc). Liquid preparations for oral administration may comprise syrups or suspensions prepared by conventional means with pharmaceutically acceptable additives such as suspending agents (e.g. hydrogenated fats, sorbitol syrup), emulsifying agents (e.g. lecithin), and preservatives. Preparations may contain buffer, salts, and flavoring agents as appropriate. Suitable examples of liquid carriers include water, alcohols, and oils containing additives as described above.

When administered, compounds are often rapidly cleared from the circulation. Thus, in an embodiment, compounds are modified by the covalent attachment of water-soluble polymers such as polyethylene glycol (PEG), copolymers of polyethylene glycol and polypropylene glycol, polyvinylpyrrolidone or polyproline, carboxymethyl cellulose, dextran, polyvinyl alcohol, and the like. Such modifications also may increase the compound's solubility in aqueous solution, and reduce immunogenicity of the compound. Polymers such as PEG may be covalently attached to one or more reactive amino residues, sulfhydryl residues or carboxyl residues. Numerous activated forms of PEG have been described, including active esters of carboxylic acid or carbonate derivatives, particularly those in which the leaving groups are N-hydroxsuccinimide, p-nitrophenol, imdazole or 1-hydroxy-2-nitrobenzene-3 sulfone for reaction with amino groups, maleimido or haloacetyl derivatives for reaction with sulfhydryl groups, and amino hydrazine or hydrazide derivatives for reaction with carbohydrate groups.

Features and advantages of the inventive concept covered by the present invention are further illustrated in the examples which follow.

### EXAMPLE 1

### Subcloning sRAGE in pBACPAK8

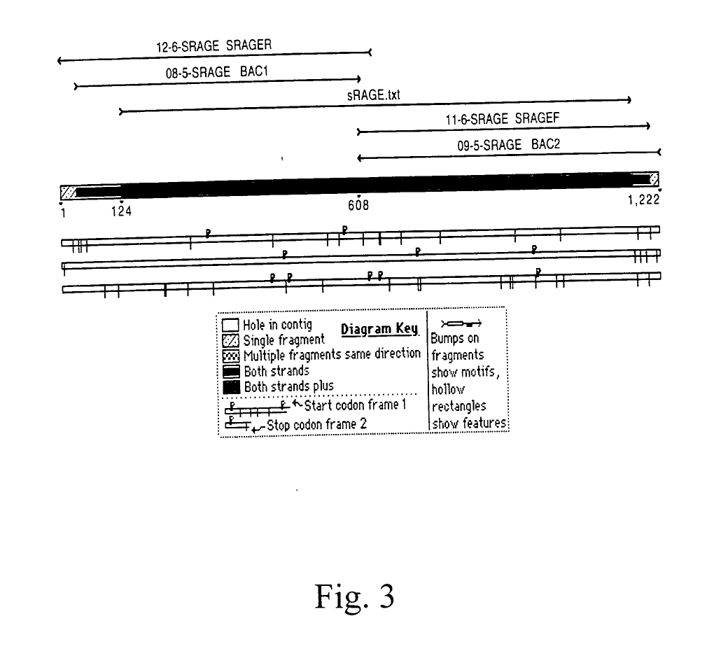

sRAGE cDNA was generated by truncation of RAGE cDNA (Neeper et al., 1992). The sRAGE cDNA was then amplified by PCR and subcloned into the multiple cloning site of EcoRI and NotI digested pBacPAK8 (Clontech, Palo Alto Calif.) as recommended by the manufacturer. The sequence of the sRAGE/pBacPAK clone was verified by DNA sequencing using the BAC1 and BAC2 primers which are recommended for sequencing inserts in pBacPAK8. These sequences are Bac1 (5′-AACCATCTCGCAAATAAATA-3′) (SEQ ID NO: 5) and Bac2 (5′-ACGCACAGAATCTAGCGCTT-3′) (SEQ ID NO: 6) and primers sRAGE-R (5′-CTCCCTTCTCATTAGGCACC-3′) (SEQ ID NO: 7) and sRAGE-F (5′-TGGGGACATGTGTGTCAGAG-3′) (SEQ ID NO: 8). A map showing the sequencing stragtegy used to verify sRAGE pBacPAK8 subclone is presented as FIG. 3.

It was found that there was a mutation at the position 7 of the sRAGE gene of Val to Ile which was corrected by PCR using forward correction primer (5′-ACGACGGAATTCTGCAGATATCATGGCAGCCGGAACAGCAGTTGGAGCC-3′) (SEQ ID NO: 9) and reverse correction primer (5′-ACGACGGAATTCCACCACACTGGACTAGTGG-3′) (SEQ ID NO: 10). The forward primer was anchored to the single base difference, extending 10 bases in the 3′ direction and 30 bases in the 5′ direction, and comprising a BamHI site in the 3′ end of the primer. The reverse primer was designed such to be antisense and complementary to the sequence immediately upstream of where the forward primer hybridizes to the original construct, but with a BamHI site on its 5′end. The correction primers were used to amplify the entire sRAGE/pBacPAK8 clone. The resulting amplicon was precipitated with ethanol, digested with BamHI and then ligated with itself to replace the mutated sequence in the clone. The sequence of the re-engineered sRAGE gene was verified by DNA sequencing as containing the human sRAGE sequence as previously described (Neeper et al.).

### EXAMPLE 2

### Insect Cell Expression

The following procedures were used to express recombinant sRAGE using the baculovirus expression vector system.

Generation of Recombinant Virus Producing sRAGE

SRAGE cDNA was subcloned into EcoRV/BglII digested pBacPAK8 and recombinant virus generated using sf9 cells as suggested by the manufacturer (Clonetech, Palo Alto, Calif.).

High Titer Virus Preparation

A culture of Sf9 cells in Sf900II (LTI)+3% FBS was initiated at a density of 1.0×10⁶ cells/ml. Culture flasks having sufficient aeration (e.g. 1 liter media in a four liter Fernbach Flask, 100 ml per 850 cm² roller bottle, or 50 ml per 250 ml Erlenmyer flask) were prepared and virus added at a multiplicity of infection (MOI) of 0.1 to 0.2 and allowed to incubate at 27° C. for 5 days with agitations at 100 rpm for the flasks and 25 rpm for the roller bottles.

After 5 days, cells were sedimented by centrifugation at 3,000×g for 10 min at 4° C., and the supernatant containing the virus filtered through a 0.2 micron sterile filter unit (S&S ZapCap or equivalent) into a sterile Nalge Bottle. The virus was then titered by plaque assay as described see below, and expressed at 1% V/V and 5% V/V for 72 h. (27° C.) using Sf9 and/or Sf21 cells at 1.5×10⁶ cells per ml. In the 1% and 5% V/V test expressions, a volumetric virus innoculum is performed while the virus titer determination is being done. In parallel, a 50 ml culture in a 250 ml flask agitated at 100 rpm was used to prepare cell pellets and supernatant for analysis of expressed proteins by sodium dodecyl sulfate polyacrylamide gel electrophoresis (SDS PAGE). Upon confirmation that the virus comprises insert DNA and expressed insert protein, the virus was stored at 4° C.

Plaque Assay

Sf9 cells were plated in Sf900II medium at a density of 1×10⁶ cells per well in a 6-well plate, and the cells allowed to attach for 1-2 hours at 27° C. Serial 10-fold dilutions of the virus stock to be titered were prepared using Sf900II medium as the diluent, and after removing the medium from each well of the 6-well plate, 1 ml of the virus dilutions were added to each well with triplicate wells per dilution. The virus was allowed to absorb for 1 hour at 27° C., and then the excess sample removed and an overlay consisting of SF900II medium (1.3×, LTI) with 1% agarose (LTI) added to each well. The gel was allowed to set a room temperature for 1 hour, and the plate transferred to a humidified chamber at 27° C. for 7 days after which the number of plaques per well were counted to determine the titer.

Cell Infection

Cultures of Sf9 or Sf21 cells in SF900II medium were initiated at an initial cell density of 2.5×10⁵ cells per ml and the cell growth monitored daily. The doubling rate was maintained in the range of 18-26 hours with a viability of >90%. When the cell density attained about 1.5-2.5×10⁶ per ml, recombinant virus was added at a multiplicity of infection (MOI) of between 0.1 and 10, as determined by an MOI time course experiment. The culture was incubated at 50% dissolved oxygen (DO) at 27° C. for 48-72 hours. Both the pellet and supernatant fluid were harvested by centrifugation at 3,000×g for 10 min at 4° C.

Generally, stirred tank bioreactors fitted with controls for oxygen and temperature are used for growth. The control of pH is not required for insect cell culture. Typical set points for baculovirus expression in bioreactors are 50% DO and 27° C. with an agitation rate of 80 rpm. These parameters are used on cultures ranging from 10-liter flasks to 150-liter stirred tanks.

MOI Time Course

To perform an MOI time course, individual flasks of Sf9 or Sf21 cells in SF900II medium were initiated at a density of 1.5×10⁶ cells per ml, and virus added at a MOI values in the range of 0.1 to 10. Cultures were incubated at 27° C. and 100 rpm for 72 hours with 2×1 ml samples of supernatant and pellets removed at 0, 24, 48 and 72 hours post-infection for characterization of expressed proteins of interest (e.g. sRAGE).

Protein Characterization

Expressed proteins can be analyzed by (1) mass spectral analysis following MALDI-TOF (matrix assisted laser desorption ionization—time of flight spectrometry); (2) ELISA assay for sRAGE; (3) SDS-PAGE eletrophoretic analysis with visualization of protein bands by Coomassie Brilliant Blue and silver staining; (4) N-terminal sequence analysis; and (5) automated C-terminal sequence analysis.

In MALDI-TOF analysis, a sample is placed within a matrix and ablated with impinging laser light. The matrix is designed to absorb the laser energy and then transfer this energy to the sample molecules. Ions are then accelerated into the MALDI flight tube (Perseptive DE Voyager Pro). The mass to charge (m/z) ratio of each ion which is detected is then reported, allowing the distribution of mass species within a given sample to be determined. Sensitivity is generally in the low fmol range for proteins.

For N-terminal sequence analysis, an aliquot of a protein sample is subjected to automated Edman degradation which reveals one residue at a time (per cycle) from the N-terminal end of the protein. The method requires that the N-terminal amino group be non-acylated so as to allow incorporation of the Edman reagent (phenylisothiocyanate). Separation of the resulting phenylthiohydration (PTH) amino acids is accomplished by an in-line RP HPLC. The entire protocol is automated (Hewlett Packard G1005A Sequencer; Hewlett Packard, Palo Alto, Calif.). Quantification is done by reference to calibration standards and sensitivity is about 5 pmol.

For automated C-terminal sequence analysis, samples are air dried onto Zitex membranes. The chemistry for this procedure is very similar to N-terminal sequence analysis, except that a 2-thiohydanton is formed following stepwise cleavage from the C-terminus of proteins/peptides. The chemistry usually reveals no more than 4 or 5 residues (Hewlett Packard 241 Sequencer; Hewlett Packard). Quantification is done by reference to calibration standards and sensitivity is about 100 pmol.

### EXAMPLE 3

### sRAGE Expression in Insect Cells

Infections were performed and cells cultured as described in Example 2. Both supernatants and cell pellets were isolated, and assayed for sRAGE ELISA with total protein measured by Bradford protein assay. In addition, an aliquot of the supernatant and pellets were subjected to SDS-PAGE.

Generally, the protocol for ELISA detection of sRAGE is as follows. A RAGE ligand (e.g. S-100b, β-amyloid, CML) is diluted to 5 μg/ml in buffer A (fixing buffer) (100 mM Na₂CO₃/NaHCO₃, pH 9.8) and 100 μl added to microtiter plate wells and allowed to incubate overnight at 4° C. to allow the ligand to become fixed to the surface of the wells. Wells are then washed 3 times with 400 μl/well buffer C (wash buffer) (20 mM Imidazole, 150 mM NaCl, pH 7.2), with a 5 second soak in buffer C between each wash. Buffer B (blocking buffer) (50 mM Imidazole pH 7.2, 5% BSA, 5 mM CaCl₂, 5 mM MgCl₂) is then added to the wells and allowed to incubate for 2 hours at 37° C. to block nonspecific protein binding sites. The blocking buffer is then aspirated from the wells, and the plate washed 3 times (400 μl/well) with buffer C, with a 5 second soak in buffer C between each wash. An aliquot from the insect culture supernatant or cell pellet containing the recombinant sRAGE is then added to each well, and incubated 1 hour at 37° C. Meanwhile, polyclonal antibody or monoclonal antibody for sRAGE (e.g. 3.0×10⁻³ mg/ml and 1.9×10⁴ mg/ml FAC, respectively), biotinylated goat F (ab′)₂ anti-mouse IgG (e.g. 8.0×10⁴ mg/ml FAC) (Biosource International, Camarillo, Calif. (TAGO)), and alkaline phosphatase labeled streptavidin (3.0×10⁻³ mg/ml FAC) (ZYMED, San Francisco, Calif.) are added to 5 ml of buffer D (complex buffer) (50 mM Imidazole, pH 7.2; 0.2% BSA, 5 mM CaCl₂, 5 mM MgCl₂) in a 15 ml conical tube and allowed to incubate 30 minutes at room temperature. FAC is final assay concentration.

The reaction mix containing recombinant sRAGE from the insect cells is then aspirated from each well, and after 3 washes with wash buffer, with a 5 second soak between each wash, the anti-sRAGE:IgG:streptavidin-alkaline phosphatase complex is added to each well (100 μl complex per well). After 1 hour at room temperature, the solution in each well is aspirated, and the wells washed 3 times, with 5 second soaks between each wash. The alkaline phosphatase substrate, para-nitrophenyl phosphate (pNPP) (1 mg/ml in 1 M diethanolamine, pH 9.8), is added and the color allowed to develop for 1 hr in the dark. After the addition of 10 μl stop solution per well (0.5 N NaOH; 50% methanol), the OD₄₀₅ is measured.

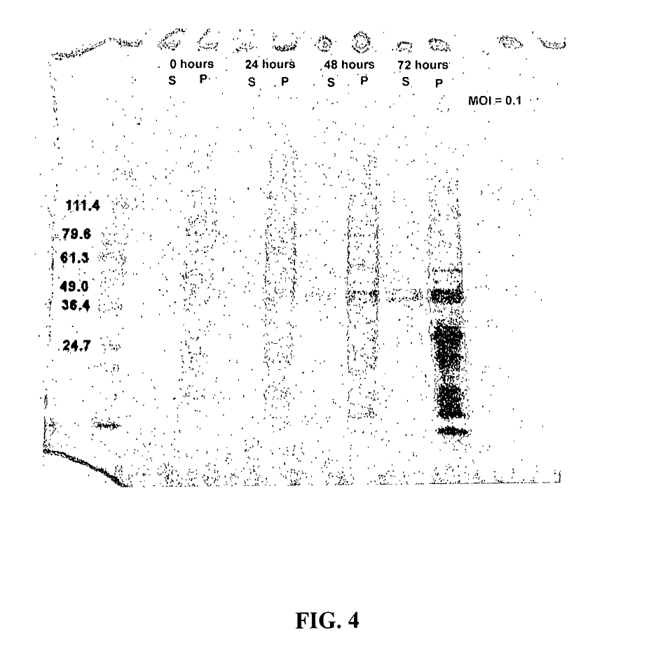

FIG. 4 shows an SDS-PAGE analysis of protein isolated from insect cell supernatants (S) and cell pellets (P). It can be seen that in the cell culture supernatants, the sRAGE band is about 36,000 Da with only minor amounts of other bands. In contrast, the cell pellet comprises multiple protein bands, with no apparent band in the sRAGE region. Thus, sRAGE is effectively secreted from the cells.

Results from an ELISA of the supernatants and cell pellets are shown in Table 1 below. The ELISA was performed essentially as described above. Protein concentration was quantified by the Bradford protein assay (Bradford, M. A., Anal. Chem., 72:238-254 (1976). It can be seen that the expression system reproducibly provides >100 mg/ml and as much as 270 mg/ml recombinant sRAGE (Table 1).

TABLE 1
Expression of sRAGE in Sf9 Insect Cells

| | Cell Supernatant | Cell Pellet |
|---|---|---|
| Experiment 1 | 278 mg/ml | 0.06 mg/ml |
| Experiment 2 | 235 mg/ml | 0.04 mg/ml |

### EXAMPLE 4

### Purification of Human sRAGE from Conditioned Insect Media

Seven liters of conditioned media were mixed in batch fashion (room temperature, 20 min) with 140 ml SP-Sepharose (Amersham-Pharmacia, Piscataway, N.Y.) (100 ml supernatant per 2 ml settled gel bed, equivalent to about 25 mg protein per ml settled gel bed) which had previously been equilibrated in 50 mM sodium phosphate buffer, pH 5.6. The suspension was mixed by means of an overhead stirrer equipped with a Teflon blade propeller.

The Sepharose gel bed was recovered by means of low speed centrifugation (5 min; 1200 rpm; ambient temperature). The supernatant was decanted and saved as the “UNBOUND” fraction. The gel bed was then washed in batch fashion with 200 ml 50 mM sodium phosphate buffer, pH 5.6 (20 min, room temperature) and then the gel bed recovered by low speed centrifugation as above. The wash was decanted and combined with the unbound fraction.

The gel bed was then washed in batch fashion with 200 ml of 50 mM sodium phosphate buffer-0.3M NaCl buffer, pH 5.6 (20 min, room temperature) and then the gel bed recovered by low speed centrifugation as above. The wash was decanted and the pH adjusted upwards to pH 7.2 (“0.3M WASH”).

The gel bed was then developed in batch fashion with 200 ml of 50 mM sodium phosphate buffer-1.0M NaCl, pH 5.6 (20 min, room temperature) and then the gel bed recovered by low speed centrifugation, as above. The desorbed fraction was decanted as the “1 M sRAGE” fraction.

After overnight at 4° C., it was observed that a precipitate formed in the 1 M sRAGE preparation. The precipitate was separated from the preparation by centrifugation and re-dissolved in pH 5.6 phosphate buffer. The pellet was only partially soluble in the low pH buffer, indicating that the pellet contained denatured protein.

All samples, including the redissolved precipitate were assayed for sRAGE by ELISA. The results of the purification are summarized in Table 2. It can be seen that that majority of the sRAGE was in the 1 M fraction, and that about 88% of the sRAGE can be accounted for in the various fractions. SDS-PAGE confirmed the ELISA, and showed a single major component of about 35 kDa in the 1M NaCl fraction.

TABLE 2
Purification of Human sRAGE from 7 liters sF9 Conditioned Media

| | | SRAGE | | |
|---|---|---|---|---|
| | Volume | (ELISA) | Percent Starting | Overall percent of |
| Sample | (ml) | total mg | Material | Starting Material |
| Conditioned | 7,000 | 1980 | 100 | 100 |
| Media | | | | |
| UNBOUND | 7,200 | 299 | 15.1 | 15.1 |
| 0.3 M | 200 | 95 | 4.78 | 19.8 |
| WASH | | | | |
| 1M sRAGE | 200 | 1359 | 68.6 | 88 |
| Redissolved | 200 | 40 | 2.0 | 90.4 |
| Pellet | | | | |

Next, several low ionic strength buffers were assessed for long-term storage of the recombinant sRAGE. Thus, the 1M NaCl sRAGE fraction was dialyzed against several buffers and the formation of precipitate monitored (Table 3). It was found that the recombinant sRAGE was stable in slightly acid buffer of 0.15 M ionic strength.

TABLE 3
Low ionic strength buffers for sRAGE

| | |
|---|---|
| 50 mM sodium phosphate, pH 5.6 | Precipitate formed |
| 50 mM sodium phosphate, 150 mM NaCl, pH 5.6 | Precipitate formed |
| 50 mM sodium phosphate, pH 6.5 | Precipitate formed |
| 50 mM sodium phosphate, 150 mM NaCl, pH 6.5 | NO PRECIPITATE |
| 10 mM sodium phosphate, 137 mM NaCl, 2.7 mM | Precipitate formed |
| KCl (PBS) pH 7.4 | |
| 50 mM Tris-HCl, 150 mM NaCl, pH 8.5 | NO PRECIPITATE |

Thus, the present invention describes methods for production of large quantities of RAGE and fragments of RAGE using an insect expression system. The use of insect expression systems for the production of membrane proteins, and membrane based proteins such as sRAGE has previously been problematic. The present invention also describes the use of recombinant RAGE produced by the methods of the invention as therapeutics for treatment of diseases caused by excess circulating AGEs. Such diseases include, but are not limited to, atherosclerosis, diabetes, kidney failure, systemic lupus nephritis or inflammatory lupus nephritis, amyloidoses, Alzheimer's disease, cancer, inflammation, and erectile dysfunction.

The invention has been described in detail with particular reference to preferred embodiments thereof, but it will be understood that variations and modifications can be effected within the spirit and scope of the invention. References cited herein are incorporated in their entirety by reference unless otherwise noted.

What is claimed is:

1. A method for high level expression of recombinant forms of the Receptor for Advanced Glycated Endproducts (RAGE) or fragments thereof comprising:
(a) subcloning a nucleotide sequence encoding RAGE or a fragment thereof into a virus;
(b) preparing a high-titer stock of recombinant virus; and
(c) infecting host cells with the high-titer recombinant virus under conditions such that pre-determined levels of RAGE or a fragment thereof is produced, wherein said pre-determined levels of RAGE comprises at least 25 mg recombinant protein per liter of culture.

2. The method of claim 1, further comprising a yield of recombinant RAGE polypeptide of more than 50 mg per liter of culture.

3. The method of claim 1, further comprising a yield of recombinant RAGE polypeptide of more than 100 mg per liter of culture.

4. The method of claim 1, further comprising a yield of recombinant RAGE polypeptide of more than 250 mg per liter of culture.

5. The method of claim 1, wherein the virus comprises the Autographa californica nuclear polyhedrosis virus.

6. The method of claim 1, wherein the host cells comprise insect cells such as Sf9 or Sf21 cells.

7. The method of claim 1, wherein the recombinant RAGE protein or fragment thereof is purified from the insect media using Sepharose.

8. The method of claim 1, further comprising infecting insect cells at a multiplicity of infection (MOI) of less than 1, and incubating the insect cell culture at a temperature of about 26-28° C. for 3-7 days to prepare high titer virus stock.

9. The method of claim 8, wherein the innoculum used to prepare the high titer stock comprises a multiplicity of infection (MOI) of 0.01 to 1.0.

10. The method of claim 8, wherein the innoculum used to prepare the high titer stock comprises a multiplicity of infection (MOI) of 0.05 to 0.5.

11. The method of claim 8, wherein the innoculum used to prepare the high titer stock comprises a multiplicity of infection (MOI) of 0.1 to 0.2.

12. The method of claim 1, wherein the nucleotide sequence encoding RAGE comprises SEQ ID NO: 1, or a sequence substantially homologous thereto.

13. The method of claim 1, wherein the fragment of RAGE subcloned for expression is the soluble, extracellular portion of RAGE (sRAGE), as encoded by the nucleic acid sequence SEQ ID NO: 2, or a sequence substantially homologous thereto.

14. The method of claim 1, wherein the fragment of RAGE subcloned for expression is the V-domain of RAGE, as encoded by the nucleic acid sequence SEQ ID NO: 4, or a sequence substantially homologous thereto.

15. The method of claim 1, wherein infecting host cells under conditions such that predetermined levels of RAGE or a fragment thereof is produced comprises the steps of:
initiating cultures of insect cells at a low density;
growing the insect cells to a preset final density;
adding the high titer virus at a MOI of less than 30; and
incubating infected cells under conditions such that a predetermined level of RAGE or a fragment thereof is produced.

16. The method of claim 15, wherein the step of infecting cells at a low density comprises cells having an initial density of no more than 0.5×10⁶ cells per ml.

17. The method of claim 15, further comprising growing the cells from an initial density of less than 0.5×10⁶ cells per ml to a final density comprising 1 to 20×10⁶ cells per ml.

18. The method of claim 1, wherein the cells are grown under conditions comprising a pre-set doubling time and viability.

19. The method of claim 18, wherein the rate of cell growth comprises a doubling rate of 10-35 hours.

20. The method of claim 18, wherein the rate of cell growth comprises a doubling rate of 15-30 hours.

21. The method of claim 18, wherein the rate of cell growth comprises a doubling rate of 18-26 hours.

22. The method of claim 1, wherein the doubling time comprises conditions such that cell viability is greater than 90%.

23. Insect cells producing recombinant RAGE or a fragment thereof according to claim 1.

24. Recombinant RAGE produced by the method of claim 1.

25. A method for high level expression of recombinant human Receptor for Advanced Glycated Endproducts (RAGE) comprising:
(a) preparing recombinant virus comprising a nucleotide sequence encoding RAGE or a fragment thereof subcloned into the Autographa californica nuclear polyhedrosis virus;
(b) preparing a high-titer stock of the recombinant virus;
(c) initiating cultures of insect cells at an initial density of less than 0.5×10⁶ cells per ml;
(d) growing the insect cells until the cell density comprises 1 to 20×10⁶ cells per ml;
(e) adding virus from step (b) at a MOI of less than 30 to the insect cells from step (d) for large scale expression; and
(f) incubating the infected culture at about 26-28° C. under conditions such that a predetermined level of RAGE or a fragment thereof is produced.

26. The method of claim 25, further comprising a yield of recombinant protein of at least 25 mg per liter of culture.

27. The method of claim 25, further comprising a yield of recombinant protein of more than 100 mg per liter of culture.

28. The method of claim 25, further comprising a yield of recombinant protein of more than 250 mg per liter of culture.

29. The method of claim 25, wherein the recombinant RAGE or fragment thereof is purified from the insect media using Sepharose.

30. The method of claim 25, wherein the nucleotide sequence encoding RAGE comprises SEQ ID NO: 1, or a sequence substantially homologous thereto.

31. The method of claim 25, wherein the fragment of RAGE subcloned for expression is the soluble, extracellular portion of RAGE (sRAGE), as defined by the nucleic acid sequence SEQ ID NO: 2, or a sequence substantially homologous thereto.

32. The method of claim 25, wherein the fragment of RAGE is the V-domain of RAGE, as defined by the nucleic acid sequence SEQ ID NO: 4, or a sequence substantially homologous thereto.

33. The method of claim 25, wherein the step of preparing the recombinant virus stock comprises infecting insect cells at a multiplicity of infection (MOI) of less than 1, and incubating the insect cell culture at a temperature ranging from about 26-28° C. for 3-7 days to prepare a high titer virus stock.

34. The method of claim 33, wherein the multiplicity of infection (MOI) used to prepare the high titer virus stock ranges from 0.01 to 1.0.

35. The method of claim 33, wherein the multiplicity of infection (MOI) used to prepare the high titer virus stock ranges from 0.05 to 0.5.

36. The method of claim 33, wherein the multiplicity of infection (MOI) used to prepare the high titer virus stock ranges from 0.1 to 0.2.

37. The method of claim 25, wherein the cells infected used for large scale expression are grown under conditions comprising a pre-set doubling time and viability.

38. The method of claim 37, wherein the rate of cell growth comprises a doubling rate of 10-35 hours.

39. The method of claim 37, wherein the rate of cell growth comprises a doubling rate of 15-30 hours.

40. The method of claim 37, wherein rate of cell growth comprises a doubling rate of 18-26 hours.

41. The method of claim 25, wherein the doubling time comprises conditions such that cell viability is greater than 90%.

42. The method of claim 25, wherein the culture used to prepare the high titer virus is grown for 3-7 days.

43. The method of claim 25, wherein the culture used to prepare the high titer virus is grown for 4-6 days.

44. The method of claim 25, wherein the culture used to prepare the high titer virus is grown for about 5 days.

45. Insect cells producing recombinant RAGE or a fragment thereof according to claim 25.

46. Recombinant RAGE produced by the method of claim 25.

47. A method for high level expression of recombinant human Receptor for Advanced Glycated Endproducts (RAGE) comprising:
(a) preparing recombinant virus comprising a nucleotide sequence encoding RAGE or a fragment thereof subcloned into the Autographa californica nuclear polyhedrosis virus;
(b) infecting insect cells at a multiplicity of infection (MOI) of about 0.1 to 0.2;
(c) incubating the insect cell culture at a temperature ranging from 26-28° C. for 3-7 days to prepare high titer virus stock;
(d) titering the virus to determine MOI;
(e) initiating cultures of insect cells at an initial density of about 2.5×10⁵ cells per ml;
(f) growing the insect cells such that the growth rate comprises a doubling time of about 18-26 hours and the cells comprise a viability of greater than 90% until the cell density comprises 1.5 to 2.5×10⁶ cells per ml;
(g) adding virus (from step (d)) at a MOI of 0.1 to 10 to the insect cells; and
(h) incubating the infected culture at about 26-28° C. for a predetermined time or until cloudy.

48. The method of claim 47, wherein the recombinant RAGE polypeptide or fragment thereof is purified from the insect media using Sepharose.

49. The method of claim 47, further comprising a yield of recombinant protein of at least 25 mg per liter of culture.

50. The method of claim 47, further comprising a yield of recombinant protein of more than 100 mg per liter of culture.

51. The method of claim 47, further comprising a yield of recombinant protein of more than 250 mg per liter of culture.

52. The method of claim 47, wherein the nucleotide sequence encoding RAGE comprises SEQ ID NO: 1, or a sequence substantially homologous thereto.

53. The method of claim 47, wherein the fragment of RAGE is the soluble, extracellular portion of RAGE (sRAGE), as defined by the nucleic acid sequence SEQ ID NO: 2, or a sequence substantially homologous thereto.

54. The method of claim 47, wherein the fragment of RAGE is the V-domain of RAGE, as defined by the nucleic acid sequence SEQ ID NO: 4, or a sequence substantially homologous thereto.

55. Insect cells producing recombinant RAGE or a fragment thereof according to claim 47.

56. Recombinant RAGE produced by the method of claim 47.

57. A method of treating human disease comprising administering the recombinant RAGE polypeptide of claim 1 in a pharmaceutically acceptable carrier.

58. The method of claim 57-, wherein the human diseases treated using the recombinant RAGE polypeptide comprise atherosclerosis, diabetes, symptoms of diabetes late complications, amyloidosis, Alzheimer's Disease, cancer, inflammation, kidney failure, systemic lupus nephritis, inflammatory lupus nepritis, or erectile dysfunction.

59. A method for inhibiting the interaction of an advanced glycosylation end products (AGEs) with RAGE in a subject, comprising administering to the subject a therapeutically effective amount of recombinant RAGE polypeptide of claim 1.

60. The method of claim 59, wherein the recombinant RAGE is administered as a therapeutically effective amount of recombinant sRAGE with an appropriate pharmaceutical so as to prevent or ameliorate disease associated with increased levels of advanced glycosylation end products (AGEs).

61. The method of claim 60, wherein the disease associated with increased levels of AGEs comprises accelerated atherosclerosis, diabetes, Alzheimer's Disease, inflammation, systemic lupus nephritis, inflammatory lupus nephritis, cancer, or erectile dysfunction.

62. The method of claim 60, wherein the effective amount of sRAGE ranges from about 1 ng/kg body weight to 100 mg/kg body weight.

63. The method of claim 60, wherein the effective amount of sRAGE ranges from about 1 μg/kg body weight to 50 mg/kg body weight.

64. The method of claim 60, wherein the effective amount of sRAGE ranges from about 10 μg/kg body weight to 10 mg/kg body weight.


SEQUENCE LISTING (10 sequences)

SEQ ID NO: 1 - DNA, 1391 bp, Homo sapiens
ggggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttgca

SEQ ID NO: 2 - DNA, 1020 bp, Homo sapiens
  CDS (1)..(1020)
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctggtctag

SEQ ID NO: 3 - protein, 339 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLV

SEQ ID NO: 4 - DNA, 336 bp, Homo sapiens
gctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacg

SEQ ID NO: 5 - DNA, 20 bp, Artificial Sequence
  Oligonucleotide primer
aaccatctcgcaaataaata

SEQ ID NO: 6 - DNA, 20 bp, Artificial Sequence
  Oligonucleotide primer
acgcacagaatctagcgctt

SEQ ID NO: 7 - DNA, 20 bp, Artificial Sequence
  Oligonucleotide primer
ctcccttctcattaggcacc

SEQ ID NO: 8 - DNA, 20 bp, Artificial Sequence
  Oligonucleotide primer
tggggacatgtgtgtcagag

SEQ ID NO: 9 - DNA, 49 bp, Artificial Sequence
  Oligonucleotide primer
acgacggaattctgcagatatcatggcagccggaacagcagttggagcc

SEQ ID NO: 10 - DNA, 31 bp, Artificial Sequence
  Oligonucleotide primer
acgacggaattccaccacactggactagtgg